(19) United States
(12) Patent Application Publication
(10) Pub. No.: US 2021/0228086 A1
(43) Pub. Date: Jul. 29, 2021

(54) NON INVASIVE SCREENING SYSTEM FOR NEONATAL HYPERBILIRUBINEMIA

(71) Applicant: S.N. BOSE NATIONAL CENTRE FOR BASIC SCIENCES, Kolkata (IN)

(72) Inventors: Samir Kumar PAL, Salt Lake (IN); Asim Kumar MALLICK, Kolkata (IN); Prantar CHAKRABARTI, Kolkata (IN); Debasis BHATTACHARYYA, Kolkata (IN)

(73) Assignee: S.N. BOSE NATIONAL CENTRE FOR BASIC SCIENCES, Kolkata (IN)

(21) Appl. No.: 17/266,221

(22) PCT Filed: May 3, 2019
(86) PCT No.: PCT/IN2019/050355

§ 371 Date: Feb. 5, 2021

(30) Foreign Application Priority Data
Aug. 7, 2018 (IN) 201831029718

(51) Int. Cl. A61B 5/00; A61B 5/024; A61B 5/145

(52) U.S. Cl. CPC A61B 5/0075; A61B 5/6898; A61B 5/14514; A61B 5/14546; A61B 5/02416; A61B 5/02438

(57) ABSTRACT

The present invention discloses a non invasive screening system for neonatal Hyperbilirubinemia based on transcutaneous bilirubin (TcB) comprising at least one nail bed transilluminating light source for penetrating subcutaneous tissue from the nail bed of neonatal subject enabling spectral analysis of circulating blood in underneath blood capillaries, a probe means cooperating with said nailbed for desired transilluminating by the selective light source held on the nail bed of the neonatal subject and reflected light collection fibre means operatively connected to spectrometric means for said spectral analysis. The spectrometric means enables identification of markers for bilirubin for desired screening the neonatal Hyperbilirubinemia in the neonatal subjects in complete range of up to 20 mg/dL bilirubin content in the circulating blood through non-invasive screening.

### FIELD OF THE INVENTION

The present invention relates to simple non-invasive screening of neonatal Hyperbilirubinemia. More specifically, the present invention is directed to develop a screening system for neonatal hyperbilirubinemia through non-invasive quantitative estimation of bilirubin level in circulating blood of neonatal subjects. The present system advantageously enables optical spectrometry-based accurate transcutaneous bilirubinometry in the neonates avoiding the effect of ambient stray light, skin tone and initiation of phototherapy interferences. The present system is particularly suitable for monitoring the bilirubin level in circulating blood of the neonates suffering from isoimmune hemolytic disease, G6PD deficiency and under phototherapy in presence of ambient light.

### BACKGROUND OF THE INVENTION

Elevated bilirubin levels in the blood of the neonates, generally known as neonatal hyperbilirubinemia or neonatal jaundice cause the yellow discoloration of the skin and other tissues of a newborn infant. Bilirubin level more than 5 mg/dL is clinical evidences of jaundice in neonates [Ref: D. J. Madlon-Kay, “Recognition of the presence and severity of newborn jaundice by parents, nurses, physicians, and icterometer,” Pediatrics 100(3), E3 (1997)]. In the first week of life, unconjugated hyperbilirubinemia is considered as a normal transitional phenomenon. According to the global statistics, jaundice is detected in almost 60% of the healthy full-term babies and 80% of the preterm babies. However, in some infants, serum bilirubin levels may rise excessively. As unconjugated bilirubin is neurotoxic so increase in bilirubin level causes acute bilirubin encephalopathyleading to either death in newborns or lifelong neurologic sequelae [Ref: N. Polley et al., “Safe and symptomatic medicinal use of surface-functionalized Mn3O4 nanoparticles for hyperbilirubinemia treatment in mice,” Nanomedicine (London, England) 10(15), 2349-2363 (2015).]. For these reasons, management of severe neonatal jaundice needs systematic evaluation of the serum bilirubin level. American Academy of Pediatrics Subcommittee on Hyperbilirubinemia has recommended that all newborns be screened before discharge for either total serum bilirubin (TSB) or transcutaneous bilirubin (TcB) measurement[Ref: M. J. Maisels et al., “Hyperbilirubinemia in the Newborn Infant Weeks' Gestation: An Update With Clarifications,” Pediatrics 124(4), 1193-1198 (2009).].

One of the earliest non-invasive method for assessment of jaundice is from human eye, which is reported as early as 1969 [Ref: L. I. Kramer, “Advancement of dermal icterus in the jaundiced newborn,” American Journal of Diseases of Children 118(3), 454-458 (1969).]. The study correlated the clinically observed cephalocaudal advancement of jaundice with the values of unconjugated serum bilirubin.

A relatively recent study systematically compared Kramer's method with the data obtained from commercially available bilirubinometers and TSB. The study grossly confirmed the findings of Kramer's [Ref: L. I. Kramer, “Advancement of dermal icterus in the jaundiced newborn,” American Journal of Diseases of Children 118(3), 454-458 (1969).], who reported a mean TSB increase of 3±2.2 mg/dL for each dermal zone for white and non-white infants. Although the transition from zone 2 to 3 was found to be associated with 0.76 mg/dL, in infants with jaundice progression to zones 3 and 4 were concluded to have risk for hyperbilirubinemia around 14% and 25% respectively [See: The Kramer Scale, FIG. 1a].

One of the pioneering works by Steven L. Jacques and co-authors [Ref: S. Jacques et al., Developing an optical fiber reflectance spectrometer to monitor bilirubinemia in neonates (1997).] on the detection of TcB using first principles of light propagation through neonatal skin was considered to be basis of development of several commercially available noninvasive bilirubinometer. The work of Steven L. Jacques and co-authors demonstrated a reasonably good correlation between TSB and TcB and anticipated the interference of pharmacokinetics of bilirubin in the neonatal blood. While Minolta JM-102 non-invasive bilirubinometer showed better performance compared to BiliChek [Ref: P. Szabo et al., “Assessment of jaundice in preterm neonates: comparison between clinical assessment, two transcutaneous bilirubinometers and serum bilirubin values,” Acta Paediatrica 93(11), 1491-1495 (2004)], obtaining institutional based calibration factor, younger/sick infants for the former and skin tones, ambient light for the latter instrument were found to have significant interference on the reliability of data from the instruments. In a recent study[Ref: F. Raimondi et al., “Measuring transcutaneous bilirubin: a comparative analysis of three devices on a multiracial population,” BMC Pediatrics 12(1), 70 (2012)] another non-invasive device BiliMed for the bilirubin screening recruited to compare with BiliChek and Minolta JM 103 and found that BiliChek and JM-103, but not BiliMed, were equally reliable screening tools for hyperbilirubinemia in multiracial neonatal population.

Although the non-invasive TcB measurement through bilirubinometry is painless and provides an instantaneous read-out of the cutaneous bilirubin concentration (TcB), limitations and opportunities of transcutaneous bilirubin measurements in neonatal subjects has been discussed in a recent study [Ref: N. Bosschaart et al., “Limitations and Opportunities of Transcutaneous Bilirubin Measurements,” Pediatrics 129(4), 689 (2012).]. It was concluded that the efficacy of the TcB bilirubinometer depended on the access of the light probe to the vascular bed. As the TcB measurement with existing bilirubinometer depends for over 99% on the contribution of extravascular bilirubin, it is a physiologically different parameter from the TSB and leads to dependence on many subject parameters including skin tones/thickness. The study suggested that the technological design of transcutaneous bilirubinometers should be improved in order to get direct access to the vascular bed in a non-invasive way for the consistency of the measured TcB with TSB.

The Indian patent 270966 discloses a conjunctival spectroscopy for the non-invasive detection of bilirubin in human subjects. However, the conjunctival spectroscopy system for the non-invasive detection of bilirubin as disclosed in Indian patent 270966 is not suitable for measuring the bilirubin level in neonatal subjects given the difficulty of accessing the conjunctiva in the neonatal subjects. Further, the measurement technique as disclosed in Indian Patent 270966 which is operable on spectroscopic signal received from the human conjunctivita is not suitable for screening neonatal Hyperbilirubinemia based on transcutaneous bilirubin (TcB).

It is thus there has been a need for developing an easy to use system for non-invasive but accurate screening of the neonatal Hyperbilirubinemia avoiding the effect of ambient stray light, skin tone and initiation of phototherapy interferences.

### OBJECT OF THE INVENTION

It is thus the basic object of the present invention is to develop a non invasive screening system for neonatal Hyperbilirubinemia based on transcutaneous bilirubin (TcB).

Another object of the present invention is to develop a non invasive screening system for neonatal Hyperbilirubinemia which would be adapted to estimate the bilirubin level in the circulating blood of the neonatal subjects avoiding the effect of ambient stray light, skin tone and initiation of phototherapy interferences.

Yet another object of the present invention is to develop a non invasive screening system for neonatal Hyperbilirubinemia which would be accurate and easy to use.

A still further object of the present invention is to develop a non invasive screening system for neonatal Hyperbilirubinemia which would be adapted to estimate the bilirubin level in the circulating blood of the neonatal subjects in real time including data acquisition, display, data analysis, generating result, making database and lastly communicate the screened bilirubin level data to remote recipient if required.

### SUMMARY OF THE INVENTION

Thus according to the basic aspect of the present invention there is provided a non invasive screening system for neonatal Hyperbilirubinemia based on transcutaneous bilirubin (TcB) comprising

at least one nail bed transilluminating selective light source for penetrating subcutaneous tissue from the nail bed of neonatal subject enabling spectral analysis of circulating blood in underneath blood capillaries;

a probe means cooperating with said nail bed for desired transilluminating by the selective light source held on the nail bed of the neonatal subject;

reflected light collection fibre means operatively connected to spectrometric means for said spectral analysis;

said spectrometric means enabling identification of markers for bilirubin for desired screening the neonatal Hyperbilirubinemia in the neonatal subjects in complete range of up to 20 mg/dL bilirubin content in the circulating blood through non-invasive screening.

In a preferred embodiment of the present non invasive screening system for neonatal Hyperbilirubinemia, the selective light source is operatively connected with the probe means through excitation fiber means;

said excitation fiber means enables transmitting of light to the nail bed for being diffused by the nail bed and transifiuminates the subcutaneous tissue illuminating the underneath blood capillaries for the spectral analysis.

In a preferred embodiment of the present non invasive screening system for neonatal Hyperbilirubinemia, the reflected light collection fibre means is configured to collect the diffused light reflected from the nail bed and send to the spectrometric means for the spectral analysis of the diffused reflected light involving generating cumulative absorbance curve corresponding to the circulating blood and therefrom calculating the bilirubin level in the circulating blood by involving the identification of markers for bilirubin for desired screening the Hyperbilirubinemia in the neonatal subjects.

In a preferred embodiment of the present non invasive screening system for neonatal Hyperbilirubinemia, the excitation fiber means comprises one or more excitation optical fibers each operatively connected to the selective light source at one end through optical coupler while at other end is exposed to the nail bed through the probe means.

In a preferred embodiment of the present non invasive screening system for neonatal Hyperbilirubinemia, the reflected light collection fibre means comprises at least one detection optical fiber operatively connected to the spectrometric means at one end while at other end is exposed to the nail bed through the probe means.

In a preferred embodiment of the present non invasive screening system for neonatal Hyperbilirubinemia, the probe means comprises a reflection probe adapted to accommodate multiple of the excitation optical fibers surrounding the detection optical fiber having their nail bed exposed ends coplanar with respect to tip of the probe;

a tubular attachment affixed on the probe tip enabling the probe tip to be held on the nail bed selectively with respect to surface of the nail bed ensuring the transmitted light from the excitation optical fibers nail bed exposed ends orthogonally fall on the nail bed only.

In a preferred embodiment of the present non invasive screening system for neonatal Hyperbilirubinemia, the tubular attachment ensures disposition of the probe tip preferably 1 cm away from the thumb nail bed surface and at 90° angle with respect to the thumb nail bed surface.

In a preferred embodiment of the present non invasive screening system for neonatal Hyperbilirubinemia, the selective light source preferably comprises tungsten halogen source adapted to generate light with uniform spectral density at wave length 470 nm and 500 nm.

In a preferred embodiment of the present non invasive screening system for neonatal Hyperbilirubinemia, the spectrometric means comprises a spectrophotometer to generate absorbance spectrum corresponding to the received diffused reflected light from the neonatal subject by converting optical spectrum array of the received diffused reflected light into wavelength array;

a computing processor to receive the absorbance spectrum and generate processed spectrum therefrom by baseline correction of the absorbance spectrum by involving dark spectrum and reference spectrum in iterative manner;

said computing processor lock the processed spectrum when absorbance of the spectrum at 630 nm falls between 0.56 and 0.6 to ensure the spectrum corresponds to reflected light collected from the light spot of constant size of diameter ˜10 mm² on the nail bed;

a memory element to temporarily store the locked processed spectrum for further processing.

In a preferred embodiment of the present non invasive screening system for neonatal Hyperbilirubinemia, the computing processor analyzes the stored processed spectrum to estimate the bilirubin level by involving

applying Gaussian fitting tool to the stored processed spectrum at different wavelengths corresponding to significant markers for oxy hemoglobin, bilirubin and highest peak in soret band and thereby generating fitted Gaussian curves for said different wavelengths;

obtaining the cumulative absorbance curve by combining the Gaussian curves;

extracting a region of interest in the cumulative absorbance curve between two wavelengths corresponding to isosbestic points;

processing the extracted region such as to obtain index value and calibrating the same with instrument index to get the bilirubin value in the circulating blood in mg/dL scale.

In a preferred embodiment of the present non invasive screening system for neonatal Hyperbilirubinemia, the spectrometric means is calibrated based on the dark spectrum and the reference spectrum whereby

the spectrophotometer generates the dark spectrum (D) corresponding to background noise in absence of an light and the reference spectrum (S) corresponding to light reflected from reference nail bed illuminated by stabilized light source for a predefined integration time without saturating the spectrophotometer; and

the computing processor corrects the baseline of the spectrophotometer generated absorbance spectrum (S) to generate the processed spectrum by involving

$$Processed spectrum = - {log}_{10} [ \frac{S - D}{R - D} ] .$$

In a preferred embodiment of the present non invasive screening system for neonatal Hyperbilirubinemia, the computing processor apply Gaussian fitting tool to the stored processed spectrum at 576 nm and 541 nm which are significant markers for oxy hemoglobin, at 470 nm which is significant marker for the bilirubin and at 415 nm which is significant marker for highest peak in the soret band.

In a preferred embodiment of the present non invasive screening system for neonatal Hyperbilirubinemia, the computing processor combine the fitted Gaussian curves for the wavelengths 576 nm, 541 nm, 470 nm and 415 nm to obtain the cumulative absorbance curve by computing

$$FC = {y}_{0} + \frac{{A}_{1}}{( {W}_{1} \times \sqrt{\frac{π}{4 \times ln 2}} ) {e}^{- 4 ln 2 {( \frac{x - 576}{{v}_{1}} )}^{2}}} + \frac{{A}_{2}}{( {W}_{2} \times \sqrt{\frac{π}{4 \times ln 2}} ) {e}^{- 4 ln 2 {( \frac{x - 541}{{v}_{1}} )}^{2}}} + \frac{{A}_{3}}{( {W}_{3} \times \sqrt{\frac{π}{4 \times ln 2}} ) {e}^{- 4 ln 2 {( \frac{x - 470}{{v}_{1}} )}^{2}}} + \frac{{A}_{4}}{( {W}_{4} \times \sqrt{\frac{π}{4 \times ln 2}} ) {e}^{- 4 ln 2 {( \frac{x - 415}{{v}_{1}} )}^{2}}}$$

wherein, A₁, A₂, A₃, A₄ are the area under the Gaussian curves and W₁, W₂, W₃, W₄ are the full width half maxima of individual Gaussian curve respectively, y₀ is offset and FC is the cumulative fitted curve.

In a preferred embodiment of the present non invasive screening system for neonatal Hyperbilirubinemia, the computing processor extracts the region of interest in the cumulative absorbance curve between isosbestic wavelengths 452 nm and 500 nm.

In a preferred embodiment of the present non invasive screening system for neonatal Hyperbilirubinemia, the computing processor process the extracted region such as to normalize absorption at 452 nm & 500 nm and extract the amplitude at 470 nm to get the index value at 470 nm.

In a preferred embodiment of the present non invasive screening system for neonatal Hyperbilirubinemia, the computing processor is operatively connect with an user interface to display the calibrated index value as the bilirubin value in the circulating blood.

According to another aspect of the present invention there is provided a method of operation of the present non invasive screening system for neonatal Hyperbilirubinemia, comprising

operatively connecting said at least one light source with the excitation fiber means to receive and transmit the light generated by the light source to the nail bed of the neonatal subject for being diffused by said nail bed and illuminate underneath blood capillaries enabling spectral analysis of the circulating blood in said underneath blood capillaries;

collecting the diffused light reflected from the nail bed though the detection fiber means to send the reflected diffused light to the spectrometric means;

spectrally analyzing the reflected diffused light by involving the spectrometric means to generate the cumulative absorbance curve corresponding to the circulating blood and therefrom calculating the bilirubin level in the circulating blood.

In the present method of operation of the present non invasive screening system for neonatal Hyperbilirubinemia, the spectral analysis of the reflected diffused light by involving the spectrometric means comprises the steps of calibrating the spectrometric means including involving the spectrophotometer to generates the dark spectrum (D) corresponding to background noise in absence of an light and the reference spectrum (S) corresponding to light reflected from reference nail bed illuminated by stabilized light source for a predefined integration time without saturating the spectrometer;

involving the spectrophotometer to generate the absorbance spectrum corresponding to the received diffused reflected light by converting optical spectrum array of the received diffused reflected light into wavelength array;

involving the computing processor to receive the absorbance spectrum and thereby generate the processed spectrum by baseline correction of the absorbance spectrum (S) based on the dark spectrum (D) and reference spectrum (R) by computing

$$processed spectrum = - {log}_{10} [ \frac{S - D}{R - D} ] ;$$

locking the processed spectrum when absorbance of the spectrum at 600 nm falls between 0.56 and 0.6 to ensure the spectrum corresponds to reflected light collected from the light spot of constant size of diameter ˜3 mm on the nail bed;

temporarily storing the locked processed spectrum in the memory element for further processing;

applying Gaussian fitting tool to the stored processed spectrum at wavelengths 576 nm and 541 nm which are significant markers for oxy hemoglobin, at wavelength 470 nm which is significant marker for the bilirubin and at wavelength 415 nm which is significant marker for highest peak in the soret band and thereby generating fitted Gaussian curves for said wavelengths;

obtaining the cumulative absorbance curve by combining the fitted Gaussian curves and computing

$$FC = {y}_{0} + \frac{{A}_{1}}{( {W}_{1} \times \sqrt{\frac{π}{4 \times ln 2}} ) {e}^{- 4 ln 2 {( \frac{x - 576}{{v}_{1}} )}^{2}}} + \frac{{A}_{2}}{( {W}_{2} \times \sqrt{\frac{π}{4 \times ln 2}} ) {e}^{- 4 ln 2 {( \frac{x - 541}{{v}_{1}} )}^{2}}} + \frac{{A}_{3}}{( {W}_{3} \times \sqrt{\frac{π}{4 \times ln 2}} ) {e}^{- 4 ln 2 {( \frac{x - 470}{{v}_{1}} )}^{2}}} + \frac{{A}_{4}}{( {W}_{4} \times \sqrt{\frac{π}{4 \times ln 2}} ) {e}^{- 4 ln 2 {( \frac{x - 415}{{v}_{1}} )}^{2}}}$$

wherein, A₁, A₂, A₃, A₄ are the area under the Gaussian curves and W₁, W₂, W₃, W₄ are the full width half maxima of individual Gaussian curve respectively, y₀ is offset and FC is the cumulative fitted curve;

extracting the region of interest in the cumulative absorbance curve between isosbestic wavelengths 452 nm and 500 nm;

processing the extracted region such as to compute deconvoluted optical density value at the wavelengths 470 and 500 nm and extract the same to get index value at 470 nm.

### BRIEF DESCRIPTION OF THE ACCOMPANYING DRAWINGS

FIG. 1 shows a schematic representation of a preferred embodiment of the present system for optical spectrometry-based transcutaneous bilirubinometry in neonates.

FIG. 1a shows the Kramer Scale (Kramer, 1969) for the visual screening of neonatal jaundice progression.

FIG. 2 shows (a) the processed spectra (difference in the elevation at 470 nm between the two curves) (b) each spectrum is fitted at four different wavelengths and (c) cumulative fit of the spectra in accordance with the present invention.

FIG. 3 shows the work flow of the present system for optical spectrometry-based transcutaneous bilirubinometry.

FIG. 4 shows the calibration curve between the instrument index value and the bilirubin value obtained from blood test.

FIG. 5 shows (a) the linear regression plot of the bilirubin measurement techniques and (b) Bland-Altman analysis of the measurement techniques.

FIG. 6 shows (a) the response of the present system to the phototherapy (b) the Bland-Altman analysis assures the detected bilirubin is differed from the biochemical technique by 1.68 units maximum or 1.44 units minimum.

FIG. 7 shows distribution of instrumental outputs for a particular subject.

### DETAILED DESCRIPTION OF THE INVENTION WITH REFERENCE TO THE ACCOMPANYING DRAWINGS

As stated hereinbefore, the present invention discloses system for optical spectrometry-based transcutaneous bilirubinometry in neonates. More specifically, the present invention is disclosing a screening system for neonatal Hyperbilirubinemia through non-invasive quantitative estimation of bilirubin level in circulating blood of the neonatal subjects.

The present system is configured to noninvasively measure whole spectrum of the blood from nail-bed using light source, optical fiber guide and spectrometric means. Instantaneous numerical analysis of the acquired spectrum (˜500 ms) starting from 400 nm to 800 nm with 1 nm interval using the present spectrometric means is found to offer several advantages over conventional non-invasive techniques including avoidance of ambient stray light, skin tone and initiation of phototherapy interferences. The measurement of the present system is comparable with gold standard TSB screening and exhibit a reasonable correlation in various physiological conditions including baby suffering from isoimmune hemolytic disease, G6PD deficiency, baby under phototherapy in presence of ambient light.

The present system is specifically configured to optically access the vascular bed under the nail bed of the neonatal subjects. The nail bed is specifically selected for the present system, as it offers several advantages in comparison to skin to access the vascular bed for the investigation TcB of a neonatal subject. The blood capillaries of the proximal nail fold run parallel to the skin surface, in longitudinal lines with longitudinal distal loops. The nail bed which is just a thin membrane in the case of neonates, acts as a perfect light diffuser in order to illuminate all the underneath blood capillaries uniformly, which is an important condition for the spectroscopic investigation of a sample using disused reflectance method. The variation of nail plate thickness compared to that of the skin is minimum across the human races. The variation of pigmentation in nail plate, particularly in neonatal subjects is also very rare as the report of melanonychia in newborns are sparse. The present system involves optical fiber guide to illuminate the nail bed and to take diffused reflectance light to a compact spectrograph for the analysis of spectral data (from 400 nm till 800 nm) in a specifically developed spectrometric technique.

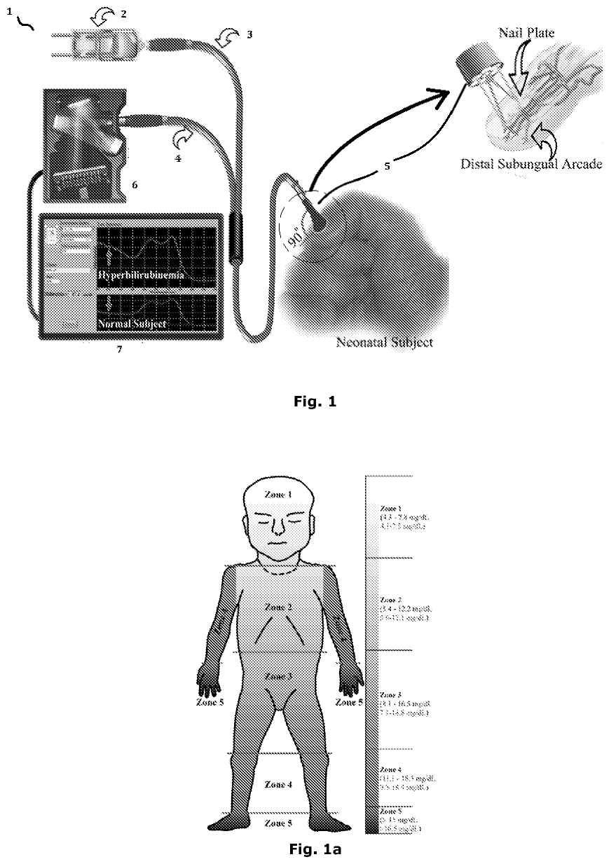

Reference is first invited from the accompanying FIG. 1 which shows a preferred embodiment of the present system. As shown in the referred FIG. 1, the present system (1) comprises a nail bed transilluminating light source (2) which is operatively connected with a probe means (5) through excitation fiber means (3). The probe means (5) is configured to cooperate with the nail bed/plate of the neonatal subject for desired transilluminating by the selective light source.

As shown in FIG. 1 and its inset, the excitation fiber means (3) transmits the light to the nail bed for being diffused by the nail bed and transifiuminates the subcutaneous tissue illuminating the underneath blood capillaries for required spectral analysis. The diffused light reflected from the nail bed is collected by the reflected light collection fibre means (4). The reflected light collection fibre means (4) send the collected diffused reflected light to the connected spectrometric means (6) for spectral analysis of the diffused reflected light based on the spectral identification of markers for the bilirubin for desired screening the neonatal Hyperbilirubinemia.

In a preferred embodiment, the nail bed transilluminating light source preferably includes tungsten halogen source (HL-2000-FHSA-LL) adapted to generate light with uniform spectral density at wave length 470 nm and 500 nm. The excitation fiber means may includes one or more excitation optical fibers each operatively connected to the light source at one end through optical coupler while at other end is exposed to the nail bed through the probe means. The reflected light collection fibre means of the present system preferably includes at least one detection optical fiber which is operatively connected to the spectrometric means at one end while at other end is exposed to the nail bed through the probe means.

The probe means of the present system comprises a reflection probe which is adapted to accommodate multiple of the excitation optical fibers surrounding the detection optical fiber. As shown in the FIG. 1 inset, the reflection probe (A) is accommodating 6 excitation fibers around 1 detection fiber having their nail bed exposed ends coplanar with respect to tip of the probe. These 6 excitation fibers are used to transmit the light from the light source to the nail bed, whereas the detection fiber is used to collect the diffused light from the nail bed and send to the spectrometric means.

As shown in the figure, the probe means also includes a tubular attachment (knurled ferrule, B) affixed on the probe tip. The aim of adding the attachment is to rest the probe tip on the thumb nail of the neonate preferably 1 cm away from the nail bed surface and to guide the incoming lights to orthogonally fall on the nail plate only. The thumb is selected nail as the target area because; it offers maximum surface area in comparison to other nails of a neonate for collecting the spectral information.

In a preferred embodiment of the present system, the spectrometric means comprises a spectrophotometer (STS-VIS) to generate absorbance spectrum corresponding to the received diffused reflected light from the neonatal subject and a computing processor to analyze the absorbance spectrum involving generating cumulative absorbance curve corresponding to the circulating blood of the neonatal subject and therefrom calculating the bilirubin level in the circulating blood by involving the identification of markers for bilirubin for desired screening the neonatal Hyperbilirubinemia in the neonatal subjects. The spectrometric means also includes a user interface e.g. windows tablet for display of the screening result and a customized operating power supply module.

The user interface embodies a graphic user interfacing means for data acquisition, display, data analysis, generating result, making database and lastly communicate the screened data to remote place if required.

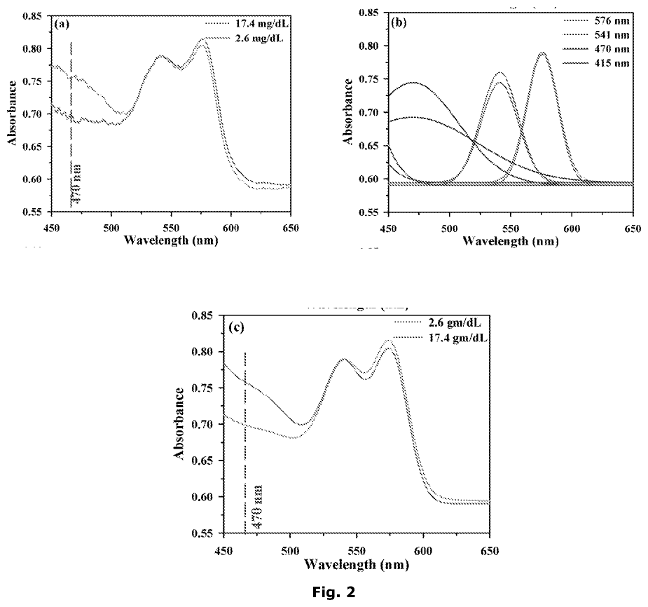

The wavelength calibration is established in the proposed system with a comparative spectral response between a normal and a jaundice subjects as represented in FIG. 2. A clear difference is visible in their spectral appearance; the contribution of yellow pigment deposited in the nail bed of the jaundice subjects is higher compared to the normal one.

### Work Flow:

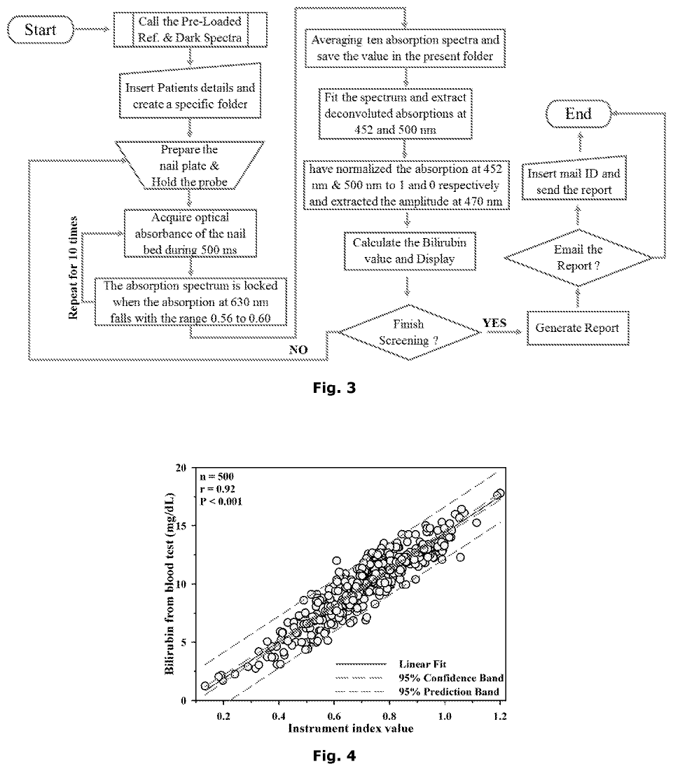

The flow of the work of the developed screening system is summarized in FIG. 3. In measurement initiation, the system is powered on and the halogen bulb based light source of the system starts glowing. After around 5 minutes the light becomes stabilized (˜7 W) and at the end of the probe tip attachment, a bright light spot is formed that penetrates the nail bed and transifiuminates the subcutaneous tissue.

Once the light source is stabilized, the probe is held on the nail plate of the neonatal subject (˜1 cm apart) so that the light beam from the tip of the probe maintains ˜10 mm² circular area of illumination and the reflected light through collection fibre is carried to the spectrophotometer. The spectrophotometer thereby generates the absorbance spectrum corresponding to the received diffused reflected light from the neonatal subject by converting optical spectrum array of the received diffused reflected light into wavelength array.

It has to be noted that the methodology adopted is completely non-invasive and non-contact assuring no external pressure on the thumb nail to force the blood out of the probing volume. The blood capillaries of the proximal nail fold run parallel to the skin surface, in longitudinal lines with longitudinal distal loops. The nail plate in infants is soft and clear, with fine longitudinal ridges which become prominent with aging. Hence, the target nail plate allows maximum light from the illumination fiber (FIG. 1) to pass through and diffuses light so that the underlying nail bed of highly vascular epithelial cells are illuminated homogeneously. The diffuse reflected light from the nail bed is persuaded through collection fiber to the spectrophotometer.

The computing processor receives the absorbance spectrum and iteratively generates processed spectrum therefrom in order to calculate absorbance of the nail bed sample in the wavelength range of 400-800 nm by baseline correction. The computing processor corrects the baseline of spectrophotometer generated blood absorption graph (S) by involving the dark spectrum (D) and reference spectrum (R) as given in the following equation.

$$Processed spectrum = - {log}_{10} [ \frac{S - D}{R - D} ] \qquad ( 1 )$$

The computing processor automatically locks the iterative generation of the processed spectrum once the absorbance of the spectrum at 630 nm falls between 0.56 and 0.6. This narrow range of absorption ensures the collection of spectral data from the constant spot size of ˜10 mm² on the target nail bed. In the diffuse reflectance spectroscopic study, the spot size of the probe light beam is an important factor to determine absorbance of an analyte for the following reasons. Firstly, the spot ensures the probe light and the tissue volume under investigation to be identical in every measurement. Secondly, the diffuse reflectance of same spot size from a reference surface is an important factor for the calculation of absorbance following Equation 1.

In the present invention, ten such locked processed spectra have been considered to generate an average spectrum in each measurement and duly saved in the specific folder. The average spectrum is called for further processing by the computing processor including fitting with four Gaussian functions (Equation 2) having peaks at 415 nm, 470 nm, 541 nm and 576 nm corresponding to the peak absorption wavelengths of soret band of hemoglobin, bilirubin and two oxyhemoglobin respectively as shown in FIG. 2b.

$$FC = {y}_{0} + \frac{{A}_{1}}{( {W}_{1} \times \sqrt{\frac{π}{4 \times ln 2}} ) {e}^{- 4 ln 2 {( \frac{x - 576}{{v}_{1}} )}^{2}}} + \frac{{A}_{2}}{( {W}_{2} \times \sqrt{\frac{π}{4 \times ln 2}} ) {e}^{- 4 ln 2 {( \frac{x - 541}{{v}_{1}} )}^{2}}} + \frac{{A}_{3}}{( {W}_{3} \times \sqrt{\frac{π}{4 \times ln 2}} ) {e}^{- 4 ln 2 {( \frac{x - 470}{{v}_{1}} )}^{2}}} + \frac{{A}_{4}}{( {W}_{4} \times \sqrt{\frac{π}{4 \times ln 2}} ) {e}^{- 4 ln 2 {( \frac{x - 415}{{v}_{1}} )}^{2}}} \qquad ( 2 )$$

The notations are as following; y₀ is offset, A₁, A₂, A₃, A₄ are the area under the curve and W₁, W₂, W₃, W₄ are the full width half maxima of individual curve respectively.

In order to deconvolute the contribution of bilirubin in the average spectrum, the peak values (415 nm, 541 nm and 576 nm) and width of the three Gaussian (34.66 nm, 29.26 nm and 36.87 nm) to be fixed during the numerical fitting. It was noted that even with free fitting of the average spectrum from all the subjects under investigation, the above parameters maintain almost constant values. As shown in FIG. 2b, the deconvoluted Gaussian curve having peak at 470 nm is consistent with that of the bilirubin absorption in the physiological condition with spectral width around 60 nm.

The Equation 2 provides a cumulative fitted curve (FC) by combining the Gaussian curves. This cumulative curve (FC) which is also called as the cumulative absorbance curve is further processed at the computing processor by way of extraction of a particular region of interest (from 452 nm to 500 nm) from the cumulative absorbance curve.

The absorption values in the wavelength range from 452 nm to 500 nm in the cumulative fitted curve as shown in FIG. 2c are extracted and considered by the computing processor for the calculation of instrument index value. The selection of the wavelength range lies on the fact that 452 and 500 nm show two isosbestic points with higher optical density in the former wavelength in the absorption spectra of oxy and deoxy-hemoglobin of whole blood of human subjects revealing insignificant interference of the oxygenation of blood at the two wavelengths. In order to calculate the instrument index value, the absorption at 452 nm & 500 nm is normalized to 1 and 0 respectively and the amplitude at 470 nm is extracted. Other attempts to deconvolute the contribution of bilirubin from the acquired data including the height and area under the curve of the deconvoluted spectrum having peak at 470 nm did not work appreciably.

The instrument index value is further converted to the bilirubin concentration using appropriate correlation plot which is required to calibrate the instrument. The regression equation is obtained from the fitting of the calibration plot as shown in FIG. 4.

After calibration, the index value is treated as the bilirubin value in mg/dL. The value is saved in a destination folder as well as displayed in the user interface. Consequently, a comprehensive medical report is instantaneously generated by the computing processor and sent to a remote recipient including the doctor and the patient through e-mail and text messaging for offline use. The user interface of the software is appropriate for use by personnel with zero or minimal medical and instrumentation knowledge.

In a preferred workflow of the present system, the computing processor calls the dark and reference spectra from a specific directory for calibration. The dark and reference spectra are required for the optical measurements because of the non-linearity of the light source's intensity, and the spectrometer's detector background noise and spectral response. To achieve sufficient signal-to-noise (S/N) ratio in the collected spectral data 500 ms integration time is maintained throughout the present study. The time needed for a detector to capture light is commonly called the integration time. More the integration time, the higher the intensity of the signal. This time needs to be adjusted to maximize the signal without saturating the spectrophotometer.

### Data Collection

A total 1033 numbers of term neonate from postnatal, neonatal intensive care unit and sick newborn care units of Nil Ratan Sircar Medical College and Hospital, Kolkata were studied for this work. The necessary ethical permissions were taken from the local medical ethics committee. 500 blood samples were taken for calibration and another 528 for validation of the instrument respectively. The information of the subjects is summarized in Table 1. To validate performance of the present system on photo therapy, 5 subjects were observed for five times at six hours interval.

TABLE 1
Statistics of the patients' information.

| | For calibration | For validation |
|---|---|---|
| Patients | N = 500 | N = 528 |
| Gestational age | 37.74 ± 1.09 | 37.70 ± 1.04 |
| | (CI: 37.64; 37.84, | (CI: 37.61; 37.79, |
| | CV: 2.90%) | CV: 2.76%) |
| Sex ratio (M/F) | 228/272 | 241/287 |
| Bilirubin | 12.13 ± 3.58 | 11.82 ± 2.64 |
| Value (mg/dL) | (CI: 11.81; 12.44, | (CI: 11.59 12.04, |
| | CV: 29.57%) | CV: 22.32%) |

Bilirubin level (21)

| | | |
|---|---|---|
| >12.9 mg/dL | N = 196 | N = 195 |
| 12.9-8 mg/dL | N = 252 | N = 295 |
| <8 mg/dL | N = 52 | N = 38 |

Other disorders

| | | |
|---|---|---|
| ABO incompatibility | N = 32 | N = 19 |
| Rhincompatibility | N = 10 | N = 2 |
| G6PD deficiency | N = Nil | N = 1 |

Treatment Undergone

| | | |
|---|---|---|
| Phototherapy | N = 102 | N = 86 |

To ensure the repeatability, ten successive readings from six different subjects were taken during validation of the system and analyzed. Before each test the infant's mother was fully explained in their native language about the experiment and utility of the study and a written consent was taken granting us permission to include their child in our study. During the tests all ethical guidelines were strictly followed.

The study was completed in four stages. In first two stages, the system was calibrated and validated respectively. In the third stage, the performance of the system during photo therapy was evaluated and lastly, examined the accuracy and precision of the same. In every stages of study, the instrument generated values were compared with the gold standard i.e. the standard biochemical method.

### Results and Discussion

### Calibration of the System:

For calibration total 500 neonates were randomly selected among which 32 were suffering from ABO incompatibility and 10 were Rh incompetence. The instrument index value from each subject was recorded during the test. Each index value was compared with the corresponding serum bilirubin value, analyzed by standard biochemical test (total serum bilirubin or TSB test). The comparisons are shown in FIG. 4. From the analysis a linear relationship was found to exist between the two procedures which can be expressed as y_{instrument_value}=15.5x_{instrument index}−1.133 with correlation coefficient (r)=0.92; P<0.001; n=500; and F=2712.

This newly developed regression equation was included in the computing processor to estimate the bilirubin level (y_{instrument_value}) from the obtained spectral information using the system.

### Validation of System:

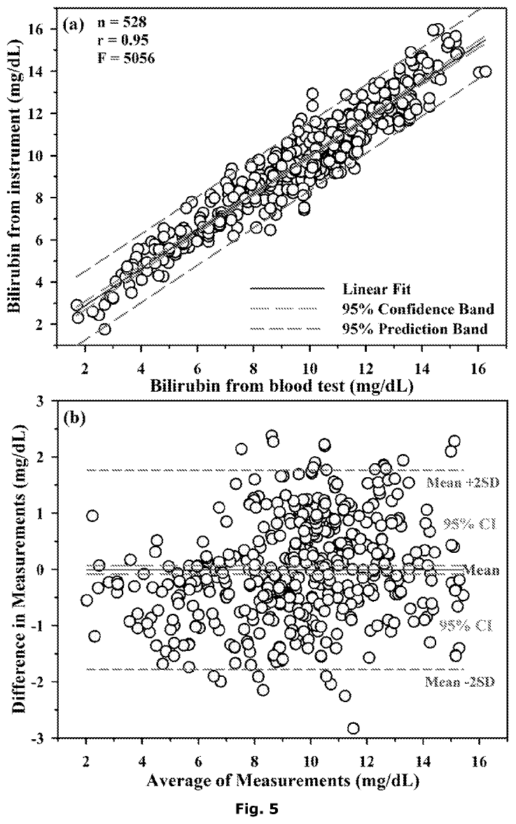

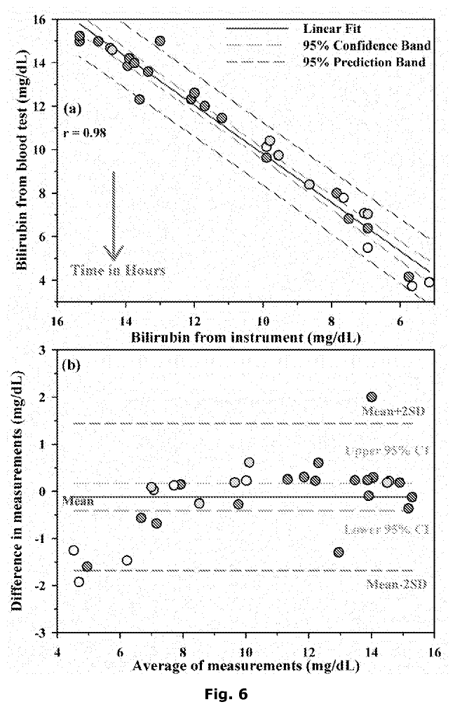

A total number of 528 subjects were selected in this part of study. In order to find the statistical significance of the instrument produced data, correlation and linear regression analyses were performed. The Bland-Altman method for assessing the agreement between the conventional biochemical technique and the present non-contact system was also tested. From the validation graph, as shown in FIG. 5a, a linear relationship was found to exist between the two procedures which can be expressed as y_{bilirubin_blood_test}=0.88x_{bilirubin_instrument}+1.12 with r=0.95; P<0.001; n=528; and F=5056. The FIG. 5a clearly shows that the system could easily screen whether the bilirubin level goes beyond the level of 12 mg/dL. The Bland-Altman analysis (FIG. 5b) ensured the agreement between two repeated measurements and the strength of the relationship between the measurement techniques. The mean value of the differences indicates a small bias of approximately −0.01 mg/dL, the limits of agreement are from −1.78 to 1.76 mg/dL and 95% confidence interval (CI) for the bias lies between −0.0850 to 0.0665. The negative bias along with CI indicates the predominant tendency of the instrument to overestimate the bilirubin levels; hence effectively avoid future errors which may cause patient harm.

In another interesting experiment the system was tested on five neonates who were prescribed for phototherapy. The data were measured on an average six hours interval. The observations, summarized in FIG. 6, shows that the device has the potential to detect the change of bilirubin level of the subjects under phototherapy. This goes to an added advantage to the present system because, the existing non-invasive instrument failed to the jaundiced infants who are receiving phototherapy as the area of skin was bleached from the phototherapy.

The Bland-Altman test shows the mean value of the differences indicates a small bias of approximately −0.12 with 95% CI between −0.4155 to 0.1676. The mean±2SD in this study also prove that the device output could vary and in 95% time the variation falls between 1.68 units less or 1.44 units greater than that of conventional biochemical method.

In another interesting experiment the system was tested by placing the probe tip both in perpendicular and in slanting orientation with respect to the nail bed. The results are shown hereunder:

TABLE 2

| | Estimated | Actual bilirubin | |
|---|---|---|---|
| Probe tip | bilirubin value | value as from | |
| disposition | by the system | bio chemical test | Error in % |
| Perpendicular | 0.64 | 0.66 | 3.03 |
| Slating | 0.71 | 0.9 | 21 |

### Repeatability of Measurements

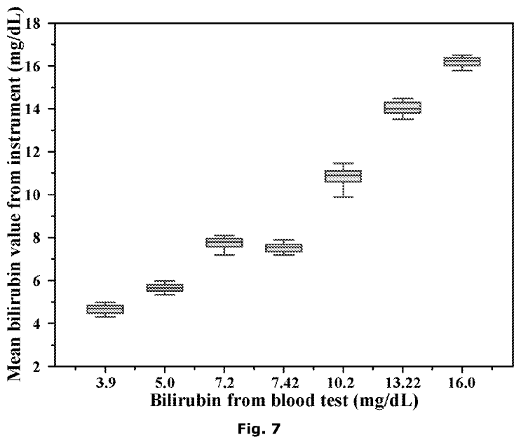

On six neonatal subjects with bilirubin concentration ranging from 3.91 mg/dL to 16.0 mg/dL the system was tested for ten successive times. Each time the same procedure was followed, by the same operator. The distributions of the data are shown in FIG. 7. The observations show that the mean coefficient of variation of less than 5.0% for the 60(6×10) tests (Table 2). Therefore it may be assumed that the marginal percentage of variation is predictable, and the proposed system is adequately precise to measure total serum bilirubin concentration levels in neonates, those are identified with clinical icterus.

TABLE 3
Precision of the proposed device

| TSB | Instrument Value (mg/dL) | | | Coefficient of |
|---|---|---|---|---|
| (mg/dL) | Mean | Mean + 2SD | Mean − 2SD | Variation (%) |
| 3.91 | 4.67 | 5.10512 | 4.24488 | 4.6 |
| 5 | 5.65 | 6.05901 | 5.25099 | 3.5 |
| 7.2 | 7.74 | 8.37386 | 7.10614 | 4.1 |
| 7.42 | 7.54 | 8.02259 | 7.05741 | 3.2 |
| 10.2 | 10.83 | 11.72963 | 9.93037 | 4.1 |
| 13.22 | 14.00 | 14.6532 | 13.3468 | 2.3 |
| 16.0 | 16.2 | 16.61825 | 15.74325 | 1.35 |

It is thus the present invention demonstrates an easy, cost effective, reliable, and portable system for measurement of bilirubin levels in neonates. The non-invasive measurement method of the present system reduces the need of frequent painful blood sampling. The setup would be useful for the initial screening as well as routine examinations. Importantly, the present system is distinct from the other existing non-invasive devices for jaundice detection (TcB) are as follows: (1) directly monitors amount of bilirubin in blood consistent with TSB with high precision up to 20 mg/dL TSB value (2) interference from other pathological conditions is minimum (2) unaffected by the phototherapy, (3) free from any mechanical attachment to the subject, (4) signal from nail bed, which is independent of skin color and (5) very limited training would be required for the healthcare provider.

One of the subtle advantages of the present system over other commercially available varieties is the detection of regression of neonatal jaundice under phototherapy (FIG. 6). Thus, progression of the neonatal jaundice may be followed either by visual check (Kramer's scale) or by noninvasive bilirubinometry, however, needs to wait till the reduction of bilirubin threshold value in the zones. Although the deposition of bilirubin pigment in the zones is well documented (refs) during the progression of neonatal jaundice, the clearance of the pigment upon regression is not reported in the literature inviting uncertainty in the detection of efficacy of phototherapy. As the present system acquire data from the nail bed which shows deposition of the pigment after 20 mg/dl, the efficacy of phototherapy can easily be detected even in the high-risk hyperbilirubinemia.

1. A non invasive screening system for neonatal Hyperbilirubinemia based on transcutaneous bilirubin (TcB) comprising:
at least one nail bed transilluminating selective light source for penetrating subcutaneous tissue from the nail bed of neonatal subject enabling spectral analysis of circulating blood in underneath blood capillaries;
a probe cooperating with said nail bed for desired transilluminating by the selective light source held on the nail bed of the neonatal subject; and
reflected light collection fibre operatively connected to spectrometric means for said spectral analysis;
said spectrometric enabling identification of markers for bilirubin for desired screening the neonatal Hyperbilirubinemia in the neonatal subjects in complete range of up to 20 mg/dL bilirubin content in the circulating blood through non-invasive screening.

2. The non invasive screening system for neonatal Hyperbilirubinemia as claimed in claim 1,
wherein the selective light source is operatively connected with the probe through excitation fiber; and
wherein said excitation fiber enables transmitting of light to the nail bed for being diffused by the nail bed and transifiuminates the subcutaneous tissue illuminating the underneath blood capillaries for the spectral analysis.

3. The non invasive screening system for neonatal Hyperbilirubinemia as claimed in claim 1, wherein the reflected light collection fibre is configured to collect the diffused light reflected from the nail bed and send to the spectrometric means for the spectral analysis of the diffused reflected light involving generating cumulative absorbance curve corresponding to the circulating blood and therefrom calculating the bilirubin level in the circulating blood by involving the identification of markers for bilirubin for desired screening the Hyperbilirubinemia in the neonatal subjects.

4. The non invasive screening system for neonatal Hyperbilirubinemia as claimed in claim 1, wherein the excitation fiber comprises one or more excitation optical fibers each operatively connected to the selective light source at one end through optical coupler while at other end is exposed to the nail bed through the probe.

5. The non invasive screening system for neonatal Hyperbilirubinemia as claimed in claim 1, wherein the reflected light collection fibre comprises at least one detection optical fiber operatively connected to the spectrometric at one end while at other end is exposed to the nail bed through the probe.

6. The non invasive screening system for neonatal Hyperbilirubinemia as claimed in claim 1, wherein the probe comprises
a reflection probe adapted to accommodate multiple of the excitation optical fibers surrounding the detection optical fiber having their nail bed exposed ends coplanar with respect to tip of the probe;
a tubular attachment affixed on the probe tip enabling the probe tip to be held on the nail bed selectively with respect to surface of the nail bed ensuring the transmitted light from the excitation optical fibers nail bed exposed ends orthogonally fall on the nail bed only.

7. The non invasive screening system for neonatal Hyperbilirubinemia as claimed in claim 6, wherein the tubular attachment ensures disposition of the probe tip preferably 1 cm away from the thumb nail bed surface and at 90° angle with respect to the thumb nail bed surface.

8. The non invasive screening system for neonatal Hyperbilirubinemia as claimed in claim 1, wherein the selective light source preferably comprises tungsten halogen source adapted to generate light with uniform spectral density at wave length 470 nm and 500 nm.

9. The non invasive screening system for neonatal Hyperbilirubinemia as claimed in claim 1, wherein the spectrometric means comprises:
a spectrophotometer to generate absorbance spectrum corresponding to the received diffused reflected light from the neonatal subject by converting optical spectrum array of the received diffused reflected light into wavelength array;
a computing processor to receive the absorbance spectrum and generate processed spectrum therefrom by baseline correction of the absorbance spectrum by involving dark spectrum and reference spectrum in iterative manner;
said computing processor lock the processed spectrum when absorbance of the spectrum at 630 nm falls between 0.56 and 0.6 to ensure the spectrum corresponds to reflected light collected from the light spot of constant size of ˜10 mm² on the nail bed; and
a memory element to temporarily store the locked processed spectrum for further processing.

10. The non invasive screening system for neonatal Hyperbilirubinemia as claimed in claim 9, wherein the computing processor analyzes the stored processed spectrum to estimate the bilirubin level by involving:
applying Gaussian fitting tool to the stored processed spectrum at different wavelengths corresponding to significant markers for oxy hemoglobin, bilirubin and highest peak in soret band and thereby generating fitted Gaussian curves for said different wavelengths;
obtaining the cumulative absorbance curve by combining the Gaussian curves;
extracting a region of interest in the cumulative absorbance curve between two wavelengths corresponding to isosbestic points; and
processing the extracted region such as to obtain instrument index value and calibrating the same to get the bilirubin value in the circulating blood in mg/dL scale.

11. The non invasive screening system for neonatal Hyperbilirubinemia as claimed in claim 1, wherein the spectrometric means is calibrated based on the dark spectrum and the reference spectrum whereby
the spectrophotometer generates the dark spectrum (D) corresponding to background noise in absence of an light and the reference spectrum (S) corresponding to light reflected from reference nail bed illuminated by stabilized light source for a predefined integration time without saturating the spectrophotometer; and
the computing processor corrects the baseline of the spectrophotometer generated absorbance spectrum (S) to generate the processed spectrum by involving
$$Processed spectrum = - {log}_{10} [ \frac{S - D}{R - D} ] .$$

12. The non invasive screening system for neonatal Hyperbilirubinemia as claimed in claim 11, wherein the computing processor apply Gaussian fitting tool to the stored processed spectrum at 576 nm and 541 nm which are significant markers for oxy hemoglobin, at 470 nm which is significant marker for the bilirubin and at 415 nm which is significant marker for highest peak in the soret band.

13. The non invasive screening system for neonatal Hyperbilirubinemia as claimed in claim 11, wherein the computing processor combine the fitted Gaussian curves for the wavelengths 576 nm, 541 nm, 470 nm and 415 nm to obtain the cumulative absorbance curve by computing
$$FC = {y}_{0} + \frac{{A}_{1}}{( {W}_{1} \times \sqrt{\frac{π}{4 \times ln 2}} ) {e}^{- 4 ln 2 {( \frac{x - 576}{{v}_{1}} )}^{2}}} + \frac{{A}_{2}}{( {W}_{2} \times \sqrt{\frac{π}{4 \times ln 2}} ) {e}^{- 4 ln 2 {( \frac{x - 541}{{v}_{1}} )}^{2}}} + \frac{{A}_{3}}{( {W}_{3} \times \sqrt{\frac{π}{4 \times ln 2}} ) {e}^{- 4 ln 2 {( \frac{x - 470}{{v}_{1}} )}^{2}}} + \frac{{A}_{4}}{( {W}_{4} \times \sqrt{\frac{π}{4 \times ln 2}} ) {e}^{- 4 ln 2 {( \frac{x - 415}{{v}_{1}} )}^{2}}}$$
wherein, A₁, A₂, A₃, A₄ are the area under the Gaussian curves and W₁, W₂, W₃, W₄ are the full width half maxima of individual Gaussian curve respectively, y₀ is offset and FC is the cumulative fitted curve.

14. The non invasive screening system for neonatal Hyperbilirubinemia as claimed in claim 11 anyone of the claims 1 to 13, wherein the computing processor extracts the region of interest in the cumulative absorbance curve between isosbestic wavelengths 452 nm and 500 nm.

15. The non invasive screening system for neonatal Hyperbilirubinemia as claimed in claim 11 wherein the computing processor process the extracted region such as to normalize absorption at 452 nm & 500 nm and extract the amplitude at 470 nm to get the index value at 470 nm.

16. The non invasive screening system for neonatal Hyperbilirubinemia as claimed in claim 11, wherein the computing processor is operatively connect with an user interface to display the calibrated index value as the bilirubin value in the circulating blood.

17. A method of operation of the non invasive screening system for neonatal Hyperbilirubinemia as claimed in claim 1, comprising:
operatively connecting said at least one light source with the excitation fiber means to receive and transmit the light generated by the light source to the nail bed of the neonatal subject for being diffused by said nail bed and illuminate underneath blood capillaries enabling spectral analysis of the circulating blood in said underneath blood capillaries;
collecting the diffused light reflected from the nail bed though the detection fiber means to send the reflected diffused light to the spectrometric means; and
spectrally analyzing the reflected diffused light by involving the spectrometric means to generate the cumulative absorbance curve corresponding to the circulating blood and therefrom calculating the bilirubin level in the circulating blood.

18. The method as claimed in claim 17, wherein the spectral analysis of the reflected diffused light by involving the spectrometric means comprises the steps of
calibrating the spectrometric means including involving the spectrophotometer to generates the dark spectrum (D) corresponding to background noise in absence of an light and the reference spectrum (S) corresponding to light reflected from reference nail bed illuminated by stabilized light source for a predefined integration time without saturating the spectrometer;
involving the spectrophotometer to generate the absorbance spectrum corresponding to the received diffused reflected light by converting optical spectrum array of the received diffused reflected light into wavelength array;
involving the computing processor to receive the absorbance spectrum and thereby generate the processed spectrum by baseline correction of the absorbance spectrum (S) based on the dark spectrum (D) and reference spectrum (R) by computing
$$processed spectrum = - {log}_{10} [ \frac{S - D}{R - D} ] ;$$
locking the processed spectrum when absorbance of the spectrum at 600 nm falls between 0.56 and 0.6 to ensure the spectrum corresponds to reflected light collected from the light spot of constant size of diameter ˜3 mm on the nail bed;
temporarily storing the locked processed spectrum in the memory element for further processing;
applying Gaussian fitting tool to the stored processed spectrum at wavelengths 576 nm and 541 nm which are significant markers for oxy hemoglobin, at wavelength 470 nm which is significant marker for the bilirubin and at wavelength 415 nm which is significant marker for highest peak in the soret band and thereby generating fitted Gaussian curves for said wavelengths;
obtaining the cumulative absorbance curve by combining the fitted Gaussian curves and computing
$$FC = {y}_{0} + \frac{{A}_{1}}{( {W}_{1} \times \sqrt{\frac{π}{4 \times ln 2}} ) {e}^{- 4 ln 2 {( \frac{x - 576}{{v}_{1}} )}^{2}}} + \frac{{A}_{2}}{( {W}_{2} \times \sqrt{\frac{π}{4 \times ln 2}} ) {e}^{- 4 ln 2 {( \frac{x - 541}{{v}_{1}} )}^{2}}} + \frac{{A}_{3}}{( {W}_{3} \times \sqrt{\frac{π}{4 \times ln 2}} ) {e}^{- 4 ln 2 {( \frac{x - 470}{{v}_{1}} )}^{2}}} + \frac{{A}_{4}}{( {W}_{4} \times \sqrt{\frac{π}{4 \times ln 2}} ) {e}^{- 4 ln 2 {( \frac{x - 415}{{v}_{1}} )}^{2}}}$$
wherein, A₁, A₂, A₃, A₄ are the area under the Gaussian curves and W₁, W₂, W₃, W₄ are the full width half maxima of individual Gaussian curve respectively, y₀ is offset and FC is the cumulative fitted curve;
extracting the region of interest in the cumulative absorbance curve between isosbestic wavelengths 452 nm and 500 nm;
processing the extracted region such as to compute deconvoluted optical density value at the wavelengths 470 and 500 nm and extract the same to get index value at 470 nm.
